(19) United States
(12) Patent Application Publication
(10) Pub. No.: US 2014/0088397 A1
(43) Pub. Date: Mar. 27, 2014

(54) Fabrication and Use of Epidermal Electrodes

(71) Applicants: Ki Chon, Worcester, MA (US); Justin Bales, Worcester, MA (US); Albert J. Swiston, Lexington, MA (US); Bersain A. Reyes, Worcester, MA (US); Hugo F. Posada-Quintero, Worcester, MA (US)

(72) Inventors: Ki Chon, Worcester, MA (US); Justin Bales, Worcester, MA (US); Albert J. Swiston, Lexington, MA (US); Bersain A. Reyes, Worcester, MA (US); Hugo F. Posada-Quintero, Worcester, MA (US)

(21) Appl. No.: 14/028,817

(22) Filed: Sep. 17, 2013

Related U.S. Application Data
(60) Provisional application No. 61/702,568, filed on Sep. 18, 2012.
(60) Provisional application No. 61/825,157, filed on May 20, 2013.

(51) Int. Cl. A61B 5/0424; B29C 39/10

(52) U.S. Cl. CPC A61B 5/0424; B29C 39/10
USPC 600/384; 600/372; 29/825; 264/102

(57) ABSTRACT

An epidermal electrode operable independently of the ambient environment, such as the presence of water, sweat, or dry conditions, and achieve a suitable impedance with the epidermal surface for transmitting electrical signals indicative of bodily physiological process such as ECG signals for heart monitoring. The hydrophobic surface mountable electrode including a flexible, conductive substrate of PDMS (Polydimethylsiloxane) with dispersed carbon black and having a substantially planar sensing area adapted for communication with an electrically sensitive surface such as a patient's skin, and a terminal for connection to a monitor circuit, the terminal having electrical continuity with the planar sensing area.

### RELATED APPLICATIONS

This patent application claims the benefit under 35 U.S.C. §119(e) of U.S. Provisional Patent App. No. 61/702,568, filed Sep. 18, 2012, entitled “ENVIRONMENTALLY INDEPENDENT ELECTRODE,” and U.S. Provisional Patent App. No. 61/825,157, filed May 20, 2013, entitled “HYDROPHOBIC ELECTROCARDIOGRAM ELECTRODES,” both incorporated herein by reference in entirety.

### STATEMENT REGARDING FEDERALLY SPONSORED RESEARCH AND DEVELOPMENT

This invention was made with government support under 5 FA7821-05-C-0002 awarded by the Office of Naval Research. The government has certain rights in the invention.

### BACKGROUND

Human physiology generates many electrical signals that may be employed for monitoring and analyzing the biological processes involved. Medical equipment is configured to receive the electrical signals for rendering and/or analyzing the signals so that medical diagnostics and conclusions may be drawn from the processed electrical signals.

In a more general context, electrical signals are often employed for various sensory and control functions in medical diagnostics and other applications. Electrodes are often employed to interface a sensory subject or an object of monitoring with a drive circuit for sensing or effecting responses from the drive circuit. Electrodes are conductive materials that facilitate an electrical interface with the subject or object of control for transmitting electrical impulses between a monitoring circuit and the subject of the sensing or control so transmitted.

### SUMMARY

An epidermal electrode transmits electrical signals from a connected subject or patient independently of the ambient environmental conditions of the subject. Conductive properties due to a carbon content provided by carbon black powder combines with a substrate medium such as PDMS (Polydimethylsiloxane) to form an electrode adapted for environmentally independent operation such as in water or gases which have electrical properties tending to interfere with conventional electrode communication. Particularly, in the case of a human epidermal electrode for sensing biological processes such as via an ECG (electrocardiogram), the environmentally independent electrode is operable to electrically couple to an epidermal (skin) surface on contact, without a need for conductive gel or suction mechanisms for maintaining an acceptable impedance (i.e. conductivity) with the epidermal surface for transmitting electrical signals along the electrode for subsequent analysis by a monitor circuit

The epidermal electrode (electrode) is therefore operable in the presence of water or sweat, and in dry environments where conventional approaches employ conductive or dielectric gel. A human subject is often analyzed using electrodes positioned and adapted to sense anatomical signals caused by physiological electrical impulses indicative of biological processes such as heart rate and brain activity. The environmentally independent electrodes disclosed herein operate on epidermal contact independently of water, sweat or conductive gel.

Configurations herein are based, in part, on the observation that conventional approaches to electronic signal monitoring of biological processes strive to achieve a definite and sustainable electrical coupling to the epidermal surface, due to the relatively low strength level of such biological signals (typically electrical impulses conducted along nerve tissue), particularly when sensing through human tissue and epidermal surfaces, which have limited conductivity. Unfortunately, conventional approaches suffer from the shortcoming that a conductive gel must often be employed between a conductive metal electrode and the epidermal (skin) surface in order to maintain a deterministic and predictable electrical coupling between the electrode and the skin, defined by an impedance of the electrode/skin interface.

Accordingly, configurations herein substantially overcome the above described shortcomings by providing an epidermal electrode operable independently of the ambient environment (such as the presence of water, sweat, or dry conditions) and achieve a suitable impedance with the epidermal surface for transmitting electrical signals indicative of bodily physiological process such as ECG signals for heart monitoring.

In further detail, configurations herein disclose an environmentally independent (i.e. wet/dry) hydrophobic surface mountable electrode including a conductive substrate having a substantially planar sensing area adapted for communication with an electrically sensitive surface, and a terminal for connection to a monitor circuit, the terminal having electrical continuity with the planar sensing area. The planar sensing area defines an impedance with a sensing surface conducive to electrical monitoring, and the conductive substrate is flexible for electrical communication upon surface placement on the electrically sensitive surface, such as the chest or wrist region of a patient being monitored.

### BRIEF DESCRIPTION OF THE DRAWINGS

The foregoing and other features will be apparent from the following description of particular embodiments disclosed herein, as illustrated in the accompanying drawings in which like reference characters refer to the same parts throughout the different views. The drawings are not necessarily to scale, emphasis instead being placed upon illustrating the principles of the invention.

FIG. 1 is a context diagram of a physiological monitoring environment suitable for use with configurations herein;

FIG. 2 is a flowchart for forming monitoring electrodes suitable for use in the environment of FIG. 1;

FIG. 3 is an example of a substantially planar electrode formed as in FIG. 2;

FIG. 4 is an example of an alternate configuration of an electrode as in FIG. 3;

FIG. 5 shows an underwater application of the electrode of FIG. 2;

FIG. 6 shows a portable configuration of a monitor circuit for the electrode of FIG. 2;

FIGS. 7A-7B show scanning electron microscope (SEM) renderings of the electrode material;

FIG. 8A-8C show a correlation of impedance to pressure of the applied electrodes;

FIGS. 9A-9B show ECG graphs of underwater divers and DCS, as in FIG. 5;

FIG. 10 shows a boxplot for the different types of electrodes; and

FIGS. 11A-11E show ECG recording on surface and underwater with wet Ag/AgCl and Carbon Black/PDMS electrodes during different conditions.

### DETAILED DESCRIPTION

Discussed below are example configurations of the hydrophobic (i.e. wet/dry) electrode operable in various ambient environments (such as underwater). In a particular configuration, an environmentally independent electrode is fabricated for operation as an underwater electrode, adapted for sensing electrical impulses despite immersion in either salt or fresh water. In alternate configurations, a “dry” electrode arrangement can eliminate the need for conductive gel to promote electrical communication between the electrode and a sensory surface, such as an epidermal (skin) surface of a human or other subject.

Further, medical sensing equipment often places electrodes on the epidermis of a subject for sensing various medical parameters, typically with a conductive gel that coats a conventional electrode in order to provide conductivity with the skin for sensing the minute electrical impulses that biological processes generate. Such so-called “dry electrodes” avoid the need for inconvenient and messy gels that often accompany such procedures.

Due to the less than ideal conductive nature of the human epidermis (skin), an impedance (electrical resistance) develops along any electrically charged member disposed on the skin surface. In a dry usage context, the claimed electrode provides an impedance at the skin surface suitable for sensing cardiac rhythms or other biological or biochemical processes. For example, cardiac electrodes need not be wetted or coated with a gel in order to provide a response to sensory current sufficient to derive output related to cardiac rhythms. Impedance, as defined herein, refers to an electrical resistance along the epidermal/electrode boundary, and is the inverse of conductivity. The impedance is sufficiently low (or unhindered) to provide for a conductivity sufficient to carry the monitored signal.

The electrodes as disclosed herein are generally a flexible, substantially planar (i.e. flat) formation that can mold to a variable annular surface such as a human body region. The example shown employs PDMS as a substrate medium and carbon black powder as a conductive medium, however other polymeric compounds and conductive substances may be employed. Further, a particular usage employed in the examples below is with an ECG taken from chest placed electrodes, however other usages may be employed, for example an electroencephalogram (EEG) or other epidermal electrode based procedure. The electrodes form an electrical coupling from mere placement on a surface, such as an epidermal application, but may also be employed for surface contact where wet conditions are expected or where a conductive gel is infeasible.

In conventional uses, carbon black is often employed with polymers for nonconductive uses such as automotive tires, composites including carbon black have not been generally associated with electrically conductive applications as provided herein.

Carbon black tends to agglomerate and form clusters when mixed or compounded with other substances. The clusters form a network, lattice or crystalline structure that, when combined in the proper density, defines an electrically conductive interconnection between the carbon black and hence, through the compound in which it is disposed. As the concentration or ratio of carbon black defines the dispersion, and therefore the distance between the carbon black clusters, conductivity often approaches a critical concentration at which the conductivity changes most rapidly.

Polydimethylsiloxane (PDMS) belongs to a group of polymeric organosilicon compounds that are typically referred to as silicones. PDMS is generally a widely used silicon-based organic polymer, and is particularly known for its unusual rheological (or flow) properties. PDMS is particularly beneficial due to the properties of being inert, non-toxic, and non-flammable.

The novel carbon black powder/PDMS composite electrode (CB/PDMS electrode) is suitable for underwater ECG monitoring due to effective performance in dry and wet conditions. Biological and medical applications of PDMS polymer are beneficial due to their simple inexpensive fabrication process in addition to their unique physical and chemical properties including superior elasticity and flexibility, non-toxicity to cells, high-permeability to oxygen, and impermeability to water. Further, their hydrophobicity makes them an interesting option for development of electrodes for ECG underwater monitoring. Low electrical conductivity of PDMS is overcome by introducing highly conductive fillers into the polymer matrix to provide continuous conductive pathways for electron migration

In contrast, in conventional approaches, a commonly used electrode for underwater ECG recording is an adhesive silver/silver chloride (Ag/AgCl) electrode surrounded by wet conductive gels. High adhesion to skin after adequate preparation makes standard wet Ag/AgCl electrodes the universal option for clinical and research application. However, shortcomings of the conventional wet Ag/AgCl electrodes include skin irritation and bacterial growth supporting in long-term recordings, gel dehydration over time, and signal degradation while sweating. Further, such electrodes have expiration dates that complicate inventory management and replacement of expired supplies. Also, their disposability increases costs of field studies on large diver cohorts, they cannot be incorporated in a neoprene protective suit, and their function tends to become inconsistent in wet and underwater conditions. Accordingly, it would be beneficial to provide a reusable, biocompatible, easily placed, and low cost ECG electrode able to be functional in a fully immersed environment must be developed.

The disclosed electrodes, therefore, need not be employed with conductive or dielectric gel as do conventional electrodes, and further are not hindered by the presence of liquids (i.e. water) on and around the sensing surface, hence they are adapted for underwater usage.

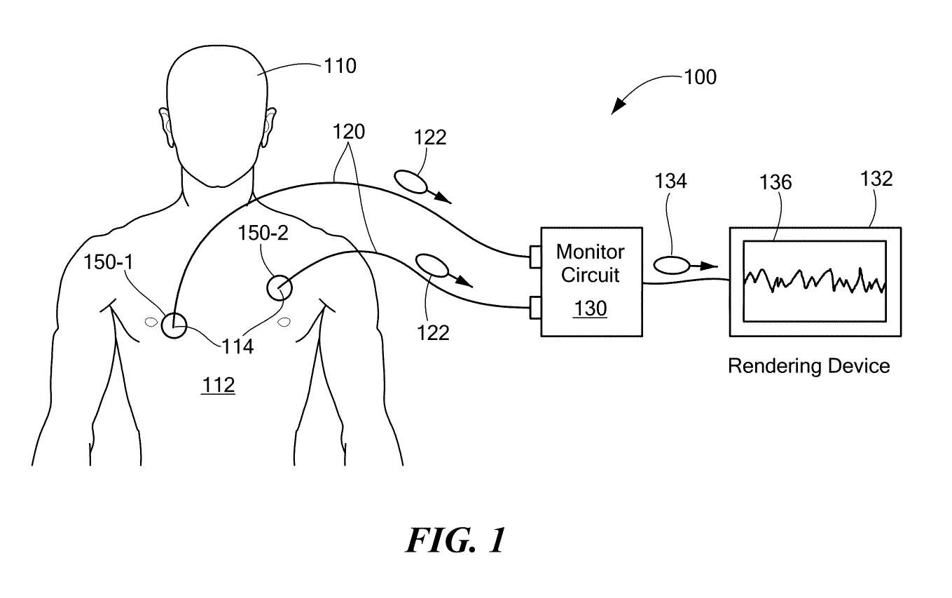

FIG. 1 is a context diagram of a physiological monitoring environment 100 suitable for use with configurations herein. Referring to FIG. 1, in an example configuration, the electrodes are employed for ECG monitoring of a human subject, or patient 110. Electrodes 150-1 . . . 150-2 (150, generally) as disclosed herein are placed on a sensing surface or area 112 for receiving electrical impulses generated by the CNS (central nervous system) and heart of the patient 110. Lead wires 120 connect terminals 114 of the electrodes 150 to a monitor circuit 130 for processing the received signals 122 defined by the electrical impulses. The processed signals 134 are rendered and/or printed on a rendering device 132 for interpretation, such as display 136. It should be noted that a plurality of lead wires 120 may be employed depending on the type of ECG and a number of leads provided for by the monitor circuit 130.

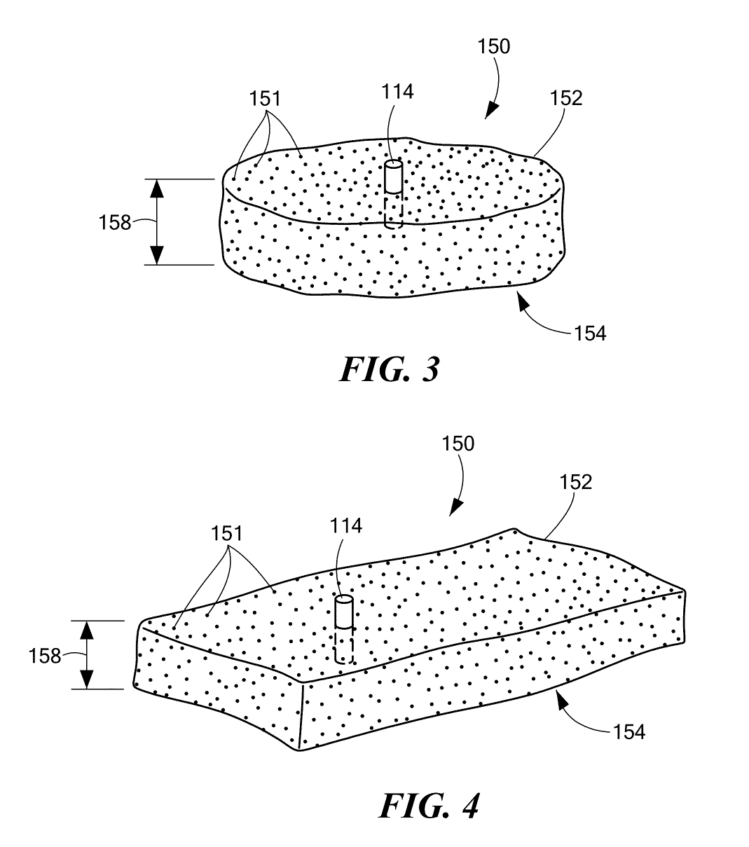

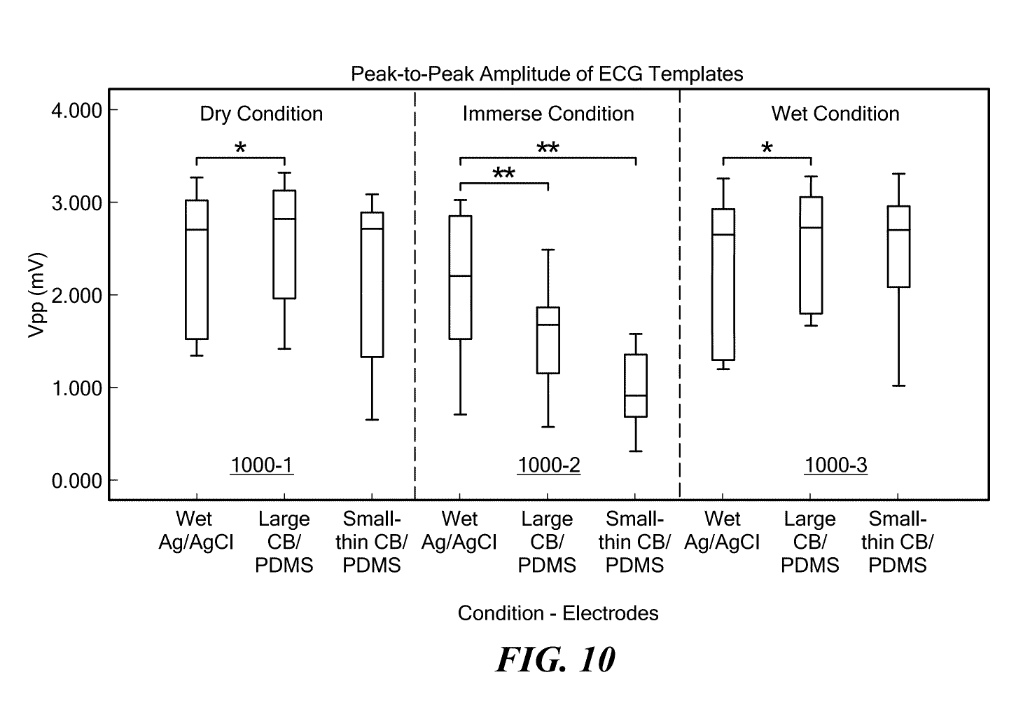

For monitoring and recording the signals 122 such as ECG signals, each electrode 150 has a sensing surface capable of carrying the electrical signals 122 sensed on the electrically sensitive surface 112 from the sensing surface to the monitor circuit 130. The sensing surface, discussed further below in FIGS. 3 and 4, is adapted to maintain a substantially constant impedance with the electrically sensitive surface 112 independently of a mounting environment, meaning that the received signals 122 are agnostic to wet or dry application, and further do not need conductive or dielectric gel, as in conventional approaches. In the example shown, the mounting environment 100 includes a human epidermis such that the electrode is unaffected by sweat and water, however alternate wet and dry environments may be employed, discussed below in FIG. 10.

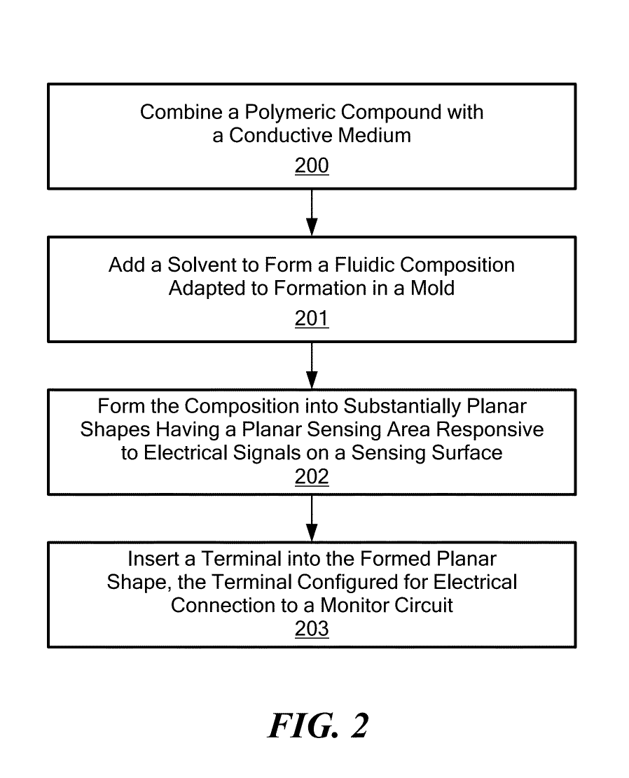

FIG. 2 is a flowchart for forming monitoring electrodes in the environment of FIG. 1. Referring to FIGS. 1 and 2, in an example arrangement, the method for fabricating a surface mount electrode operable in wet or dry conditions includes, at step 200, combining a polymeric compound with a conductive medium. The polymeric compound forms a flexible substrate that conforms to the sensing surface in wet, dry, gaseous or other conditions that may tend to interfere with convention electrodes. The conductive medium, such as a carbon black powder, becomes dispersed in the substrate material and forms clusters, or agglomerations, such that an electrical charge is transferred between the particles of the conductive medium. A solvent is added to form a fluidic composition adapted to formation in a mold, as depicted at step 201, allowing the substrate dispersion to be poured, formed and/or shaped. The composition is formed into substantially planar shapes having a planar sensing area responsive to electrical signals on a sensing surface, as depicted at step 202. A terminal 114 for electrical connection is inserting into the formed planar shape, or placed in a mold prior to pouring, such that the terminal is configured for electrical connection to a monitor circuit, as depicted at step 203. The terminal 114 is generally a rigid conductor for facilitating an electrical connection, and is surrounded by a sufficient area of the composition to conduct the electrical signal from the electrode 150 substrate. In the example arrangement, the terminal takes the form of a snap connector for mating with a complementary receptacle on an end of the lead wires 120.

In an alternate configuration, electrode fabrication includes dissolving a predetermined quantity of Trifluropropyl POSS (FPOSS) into 50 ml of Asahiklin, an adding the FPOSS solution to the composition effects a surface treatment of nanostructured particles. In practice, it has been found that a predetermined quantity of FPOSS is in the range of 5-50 mg FPOSS per 50 ml of Asahiklin imparts nanostructured particles, in this case Trifluropropyl POSS (Hybrid Plastics, FL0578), a super hydrophobic surface can be obtained

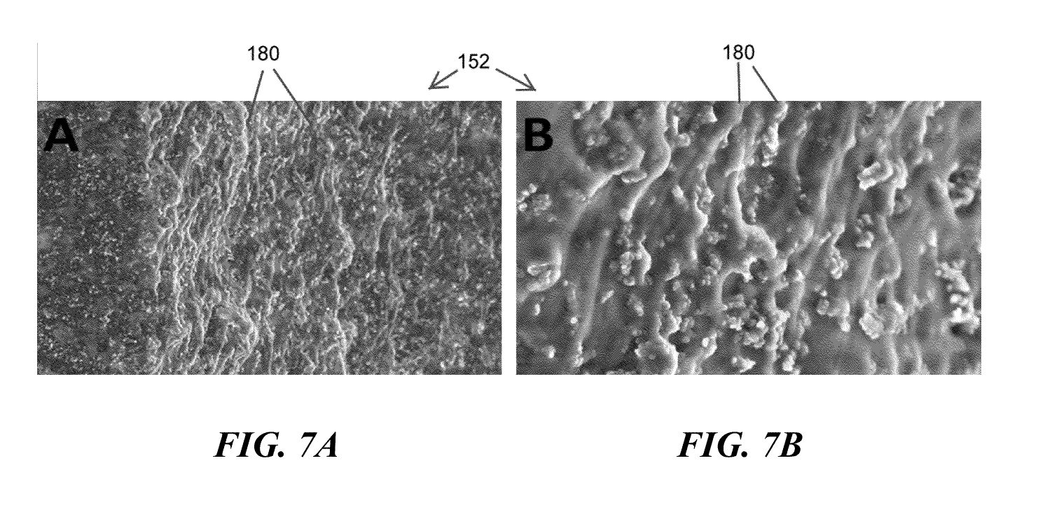

FIGS. 3 and 4 are examples of a substantially planar electrode formed as in FIG. 2. Referring to FIGS. 1, 3 and 4, the surface mountable electrode 150 includes a conductive substrate 152 having a substantially planar sensing area 154 adapted for communication with an electrically sensitive surface. In the example shown, the conductive substrate 152 includes a composition of a polymer and conductive particles 151. Carbon black is chosen such that the composition is adapted to form conductive agglomerations based on a density of the dispersed conductive medium, in which the polymeric compound includes PDMS and the conductive medium is carbon black powder. The graphs below in FIGS. 7-10 depict measurements taken where the conductive substrate 152 includes a dispersion of carbon black, such that the carbon black achieves a density based on a predetermined concentration defined by an ability to conduct an electrical signal through the substrate 152. Other configurations may employ alternate polymers and/or conductive mediums.

The terminal 114, for connection to a monitor circuit, is molded or integrated in the substrate 152, such that the terminal 114 has electrical continuity with the planar sensing area 154. The terminal 114 is mounted to the substrate 152 for connection to the control (monitor) circuit 120, in which the control circuit is responsive to the electrode 150 and the electrode is adapted to sense electrical signals unaffected by liquid presence on the substrate 152. The planar sensing area 154 therefore defines an impedance with the sensing surface 112 conducive to electrical monitoring, and the conductive substrate being flexible for electrical communication upon surface placement on the electrically sensitive surface (i.e. epidermis) of a patient. As indicated above, conventional approaches require gel for providing an acceptable impedance between the sensing surface 112 and the planer sensing area 154 (sensing area). In contrast, with the disclosed electrodes 150, the defined impedance is independent of environmental conditions on the sensing surface 112. The defined impedance is substantially constant in wet or dry ambient conditions on the sensing surface 112. The impedance of the formed electrode 150 is defined by a thickness 158 and area of the sensing area 154.

Carbon black, employed as the conductive medium of particles 151, is formed by combusting heavy oils in a furnace, and it has proven to be a versatile functional filler due to dispersion, structure, consistent particle size, and purity. In contrast to carbon nanotubes where the homogenous dispersal in thick PDMS is challenging, carbon black particles have been found to be easy to mix with PDMS gel and uniformly distributed in PDMS. The conductivity of CB/PDMS composites have been found to increase rapidly beyond a threshold concentration (circa 10 wt %). The carbon black content increment forms a conductive network throughout the isolation matrix that decreases the electrical resistivity. Distance between particles decreases with carbon black concentration increment, resulting in a facilitated transport of electrons. However, it should be noted that when the concentration of the solid conducting phase is too high, the mechanical characteristics of the composite no longer resemble those of PDMS and it becomes stiff and easy to break

FIG. 4 shows an example of an alternate configuration of an electrode as in FIG. 3. In both FIGS. 3 and 4, the conductive substrate is a homogeneous structure having a planar contact surface 154 and an integrated electrode, such that the homogeneous structure is formed around the terminal 114 for passing electrical signals from the planar contact surface through the electrode 150 to the control circuit 130. Since the defined impedance of the substrate 152 is proportional to the area of the planar surface, such that the strength of a sensed electrical signals increases with the area, the rectangular shape of FIG. 4 may provide a greater area on the sensing surface 112 depending on space considerations.

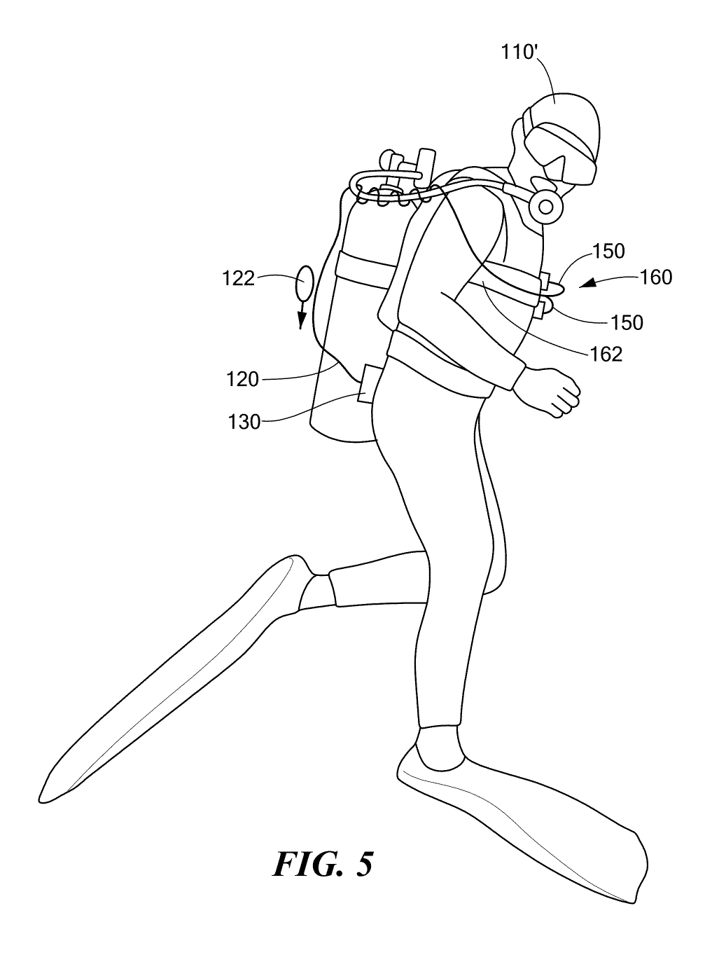

FIG. 5 shows an underwater application of the electrode of FIG. 2. Underwater divers often suffer from irregular cardiac (heart) anomalies due to respiratory side effects of the underwater breathing equipment. The environmentally independent electrodes are defined as hydrophobic electrocardiogram electrodes adapted for a waterborne environment and configured to sense cardiac rhythms in an underwater setting where conventional electrodes would have their true reading affected by the aqueous presence.

Referring to FIGS. 1 and 5, in particular configurations, the mounting environment 100 is submerged underwater and the electrode is affixed to a cardiac region of a subject 110′, in which the cardiac region is for transmitting cardiac rhythms 122 to the sensing or monitor circuit 130 via the electrode 150. A strap 162 or other mechanism affixes the electrodes 150 to underwater divers and receive the signals 122 indicative of a respiration of the underwater diver. Note that the electrodes 150 are shown for visibility, and would actually be adhered on the skin surface underneath any wetsuit or diving gear worn by the subject 110′.

In a particular configuration, the approaches herein employ the monitor circuit 130 to monitoring the signals 122 received from the underwater divers for detecting symptoms of decompression sickness (DCS)., i.e. bloodstream borne gas bubbles. DCS is characterized by a variance of the heart rate impulses, such that computing a heart rate variability (HRV) based on variances of distance between the peaks of the monitored signals may be employed to identify DCS based on the variances.

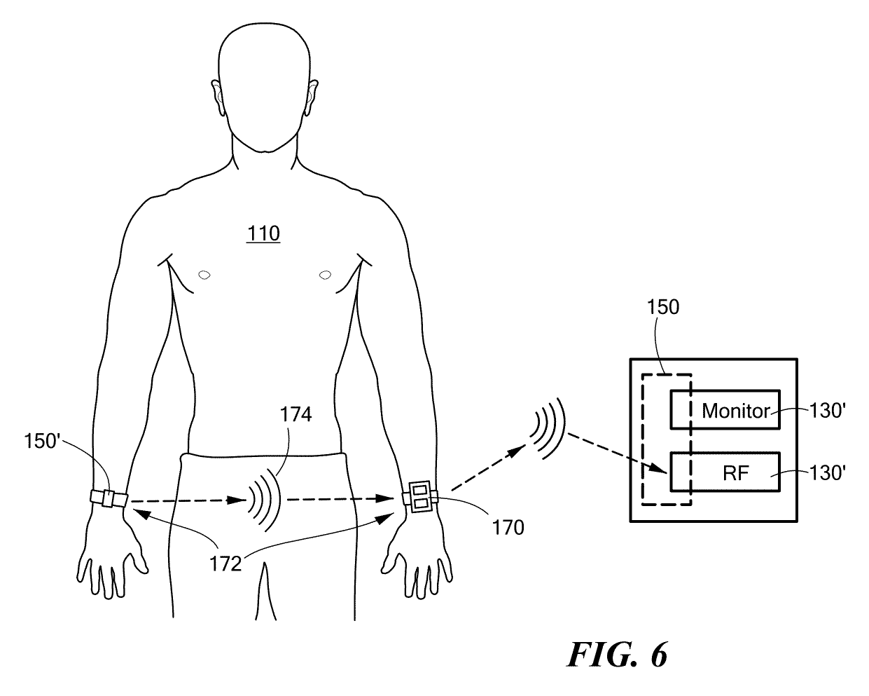

FIG. 6 shows a portable configuration of a monitor circuit for the electrode of FIG. 2. Cardiac monitoring practices often require extended monitoring periods during which the patent 110 remains connected to an adequate monitoring apparatus. Conventional approaches require a bulky harness for deploying the electrodes and adequate monitoring and recording capability. In the approach of FIG. 6, a wrist-based approach employs a portable wristwatch configuration for coupling the electrodes 150 with the wrists of the patient 110, such that the monitored signals detect and define continuous measurement of paroxysmal atrial fibrillation (abnormal heartbeat).

In the approach of FIG. 6, an electrode 150 is affixed to an underside of a wristwatch appliance 170 in communication with the wrist epidermis 172 for sensing cardiac signals. A complementary electrode 150′ is affixed on an epidermis of an opposed wrist for sensing a complementary signal, as two signals are received and compared for analysis. The appliance 170 performing continuous monitoring of the cardiac signals obtained via the electrode 150 and complementary electrode 151 by onboard monitor electronics 130′. An RF link 174 invokes an RF module 176 in the appliance 170 for coordinating and synchronizing the different signals received at each wrist 172. The RF link 174 may take any suitable form, such as Bluetooth, WiFi, RFID or other mechanism for transferring timing information. The monitored signals are stored in the wristwatch appliance 170 for subsequent analysis and/or rendering, by any suitable mechanism such as non-volatile flash memory, to avoid power supply compromise of the stored information. Such a wrist based approach is particular useful for providing continuous measurement for detecting paroxysmal atrial fibrillation or irregular heartbeat.

FIGS. 7A-7B show scanning electron microscope (SEM) renderings of the electrode material; In FIGS. 7A and 7B, the microstructure of CB/PDMS electrodes was observed via high-vacuum SEM micrographs after freeze-fracture. FIG. 7A is a rendering with scale bar=1000 nm, and in FIG. 7B, the scale bar=300 nm. Deposition of the carbon black particles inside the elastomeric matrix is clearly seen as agglomerations 180 that define a conductive path through the substrate 152.

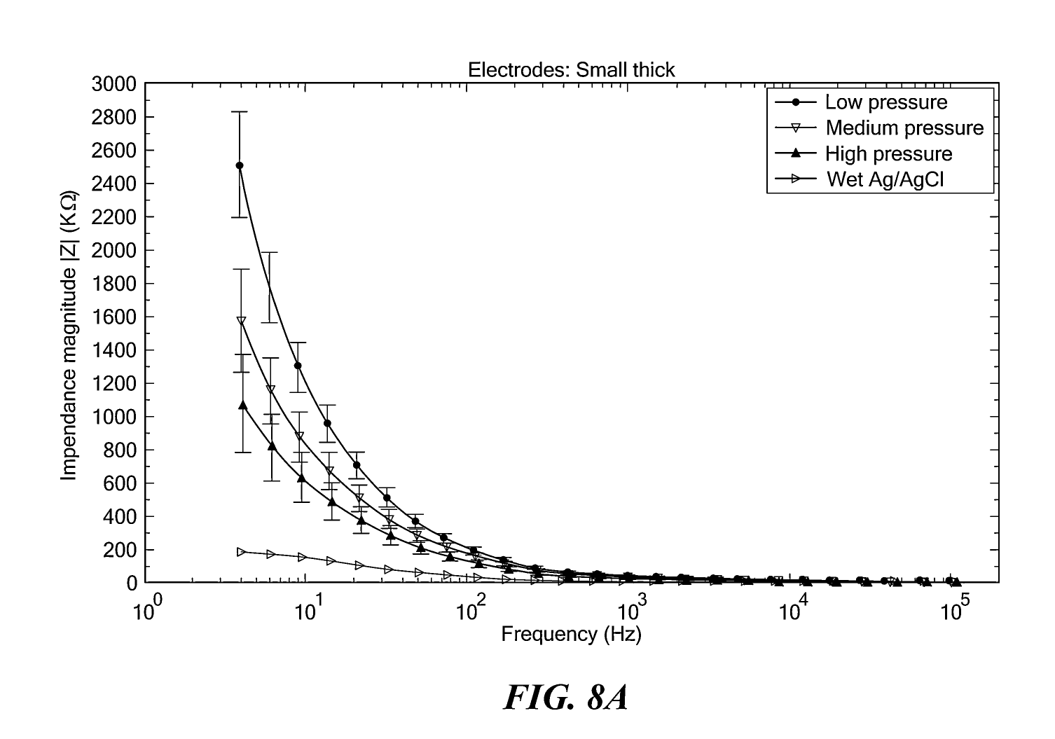

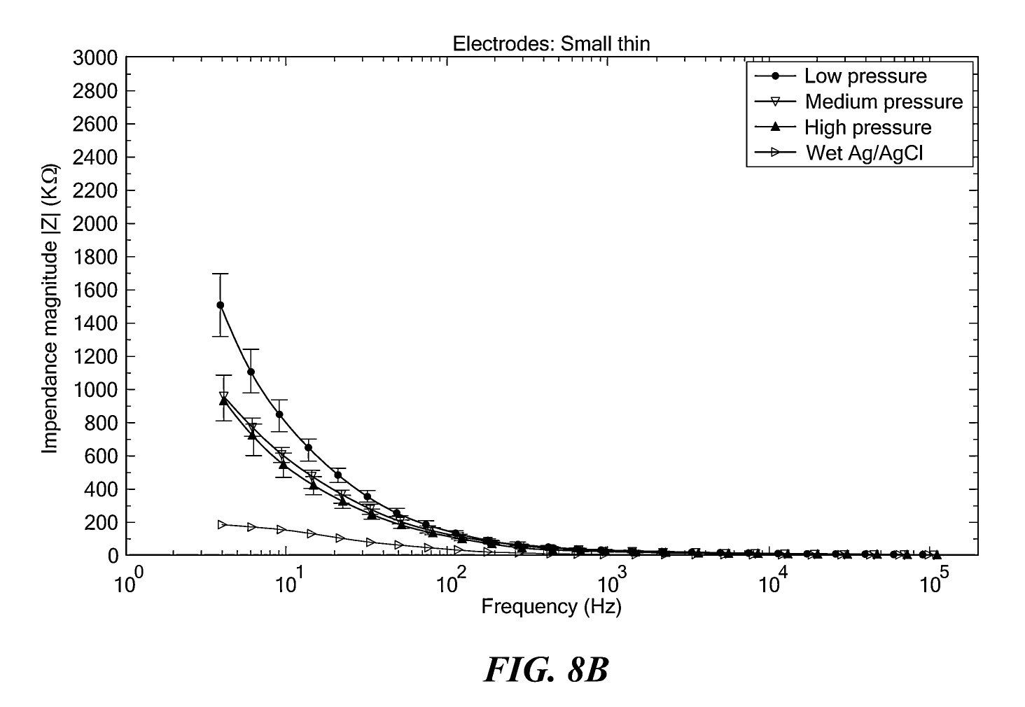

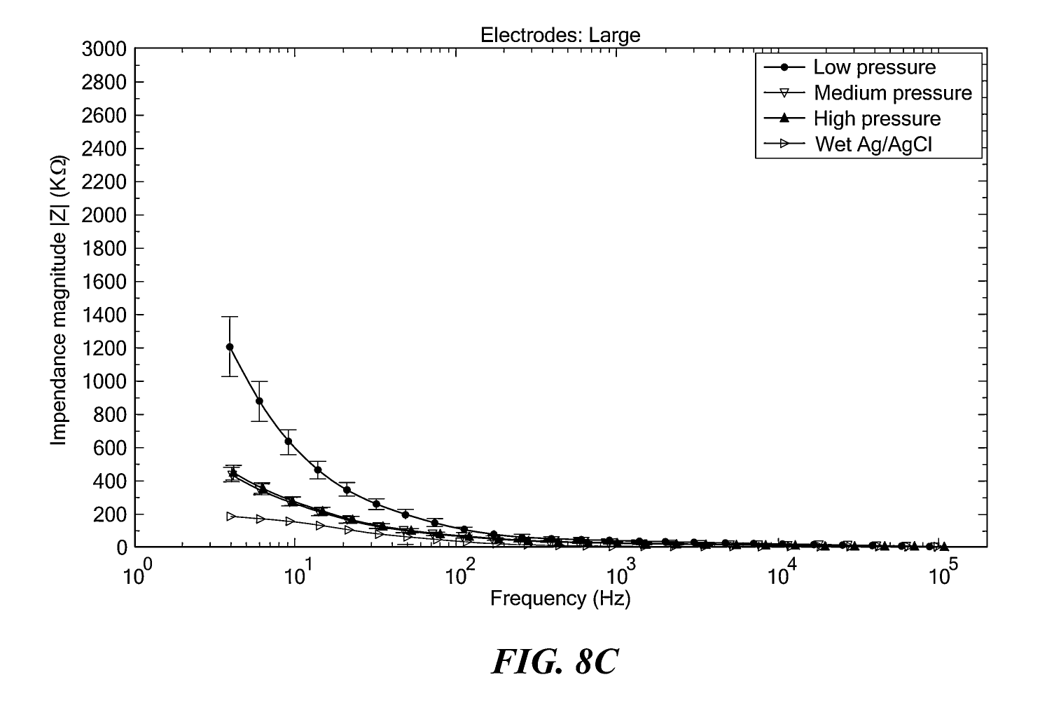

FIG. 8A-8C show a correlation of impedance to pressure of the applied electrodes. Referring to FIGS. 3, 4 and 8A-8C, the impedance magnitude IZI results may be plotted against frequency. Shown is the pressure dependence of electrode-skin impedance for the CB/PDMS electrodes of same dimensions. As indicated above, impedance (conductivity) varies with the area of the sensing surface 154 and the thickness 158. FIG. 8A shows the impedance for smaller, relatively thick electrodes 150. FIG. 8B shows a small, thinner electrode. FIG. 8C depicts an electrode having a larger area, such as the rectangular configuration of FIG. 4. In each case, the impedance is dependent on the applied pressure and its value decreases with increasing frequency, although for the two thinner electrodes (2 mm thickness) there is only a slight difference between medium and high pressure levels.

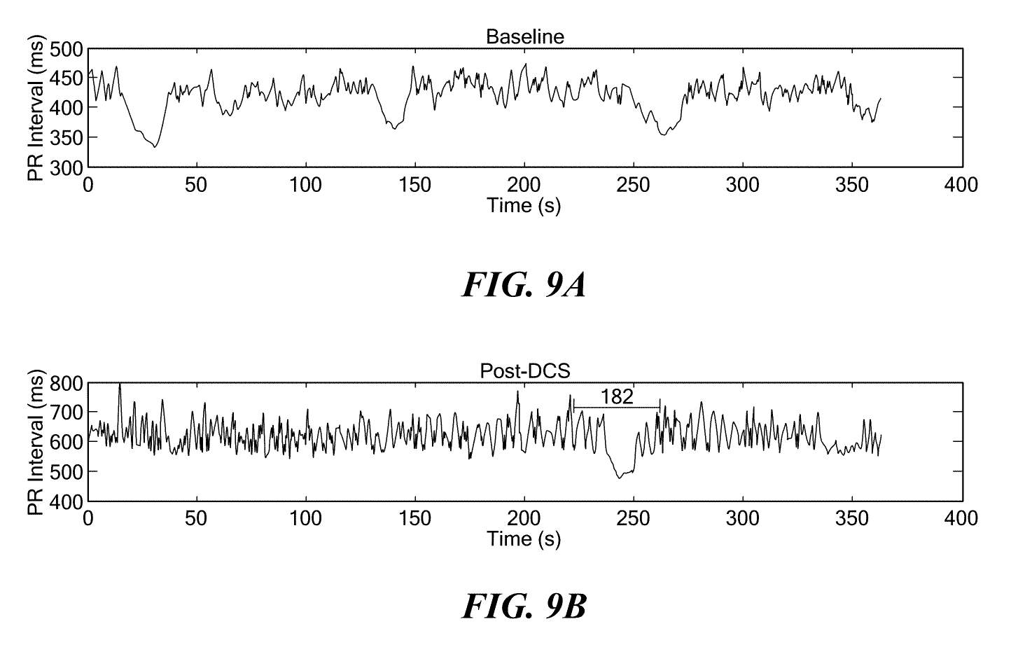

FIGS. 9A-9B show ECG graphs of underwater divers and DCS, as in FIG. 5. In a diver exhibiting a DCS condition (FIG. 9B), the heart rate variability 182 (distance between peaks) is increased. Conventional electrodes do not exist to record an ECG signal that would allow for the detection of HRV.

FIG. 10 shows a boxplot with the results obtained for the peak-to-peak amplitude for each type of electrode during each experimental condition-dry 1000-1, immersed 1000-2, and wet (post-immersion) 1000-3. The amplitude obtained with the large CB/PDMS electrode was found to be statistically significant higher compared to the wet Ag/AgCl amplitude (p<0.05) during the dry condition. Both sizes of CB/PDMS electrodes produced lower amplitudes of ECG templates than the wet Ag/AgCl electrode during the immerse condition (p<0.05). For the wet (post-immersion) condition, similar statistical results to the dry condition were obtained.

For the immersion and post-immersion conditions, amplitude attenuation/gain of ECG templates with respect to the initial pre-immersion period was computed by dividing the peak-to-peak amplitude of ECG template of the non-dry conditions by the corresponding amplitude obtained during the dry condition with the same type of electrode. Amplitude reduction/gain results from CB/PDMS electrodes were compared to those from wet Ag/AgCl electrodes, and statistically significant lower reduction was found for both sizes of CB/PDMS when compared to the wet Ag/AgCl during the immersion condition (p<0.05); for the wet condition, statistically significant higher gain was found for the small-thin CB/PDMS electrodes when compared to the wet Ag/AgCl (p<0.05).

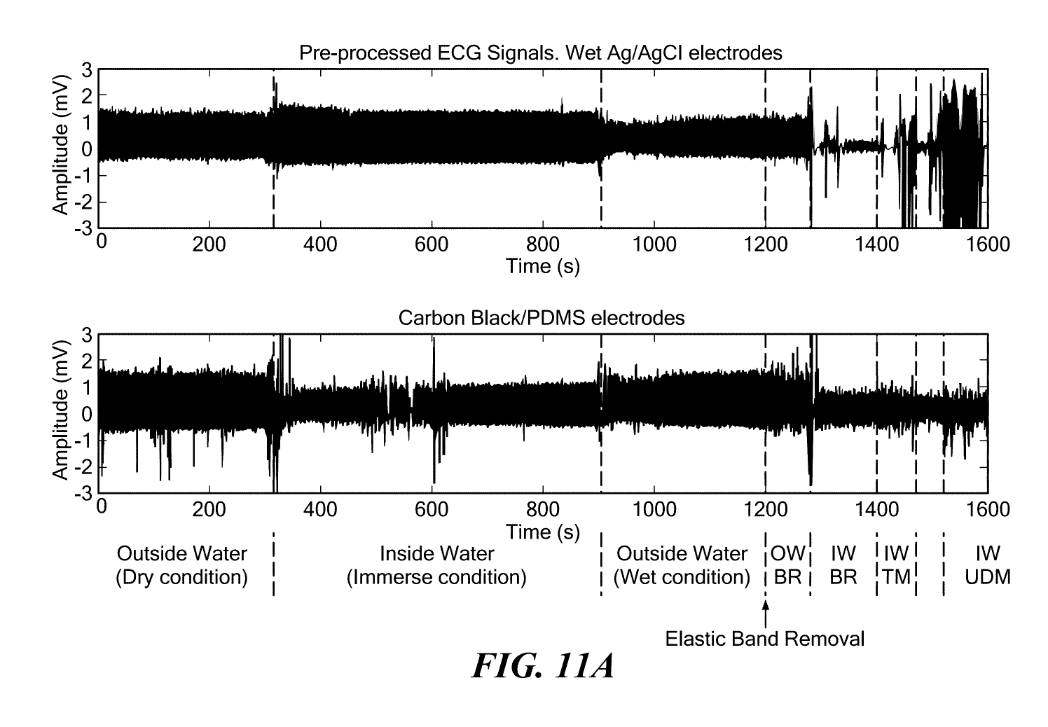

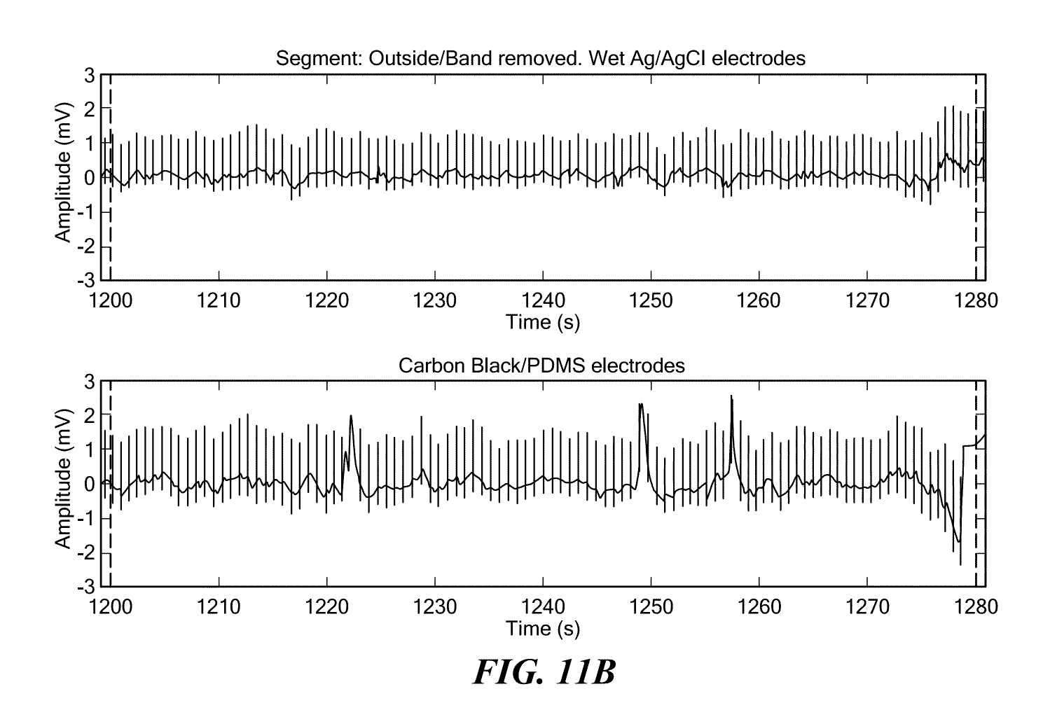

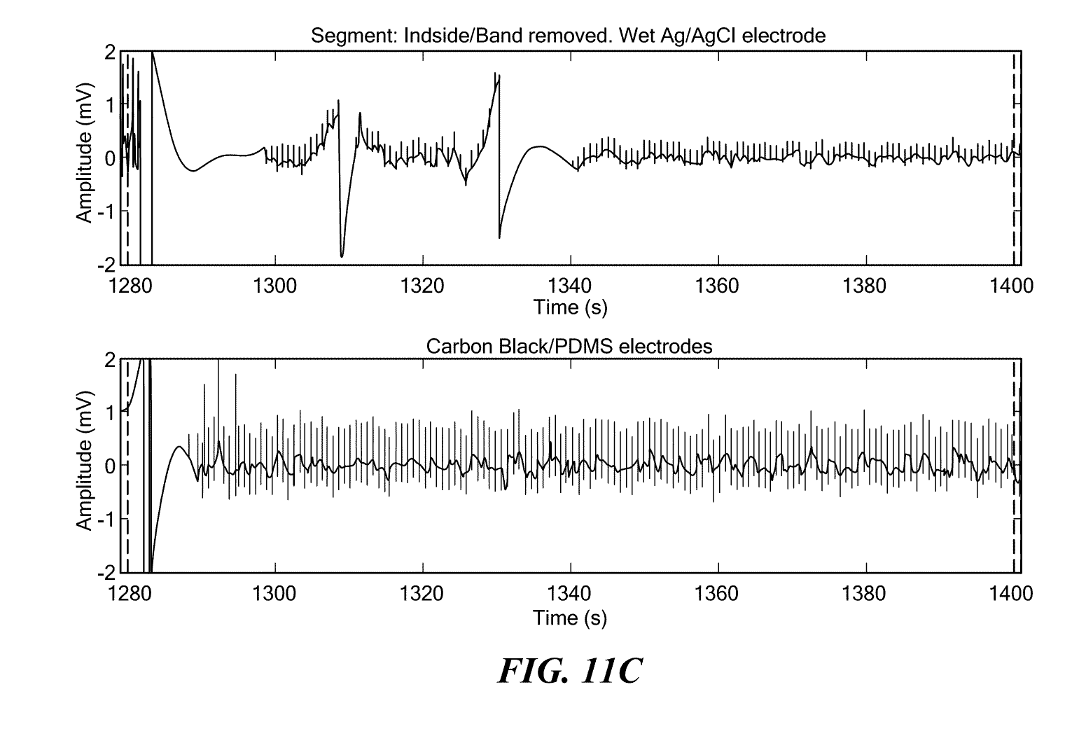

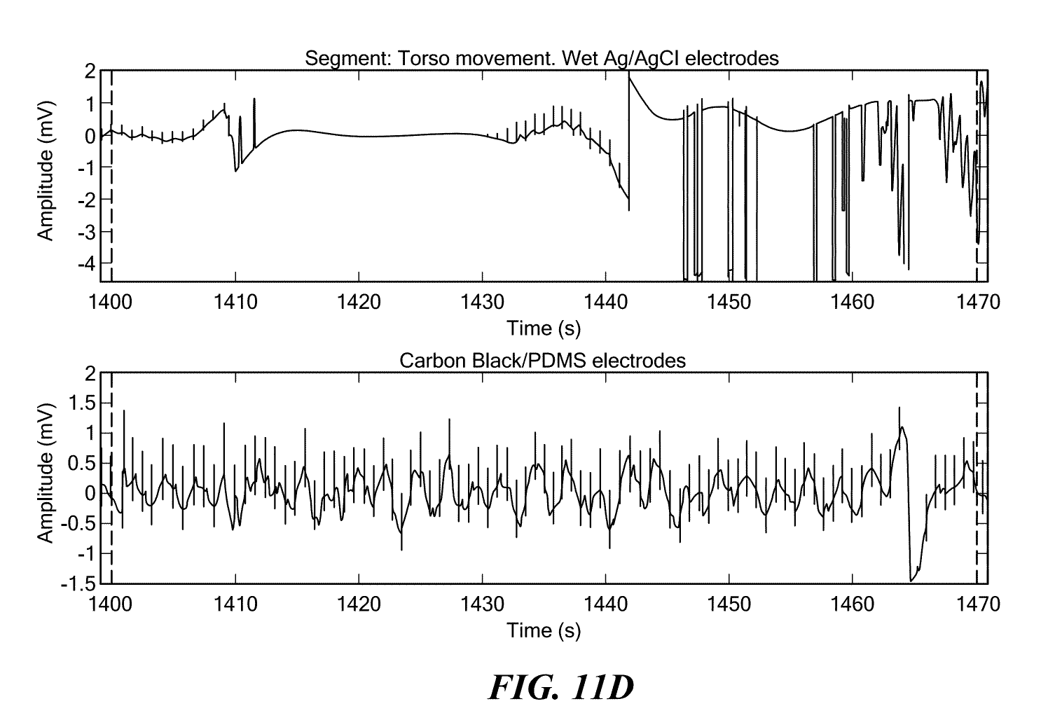

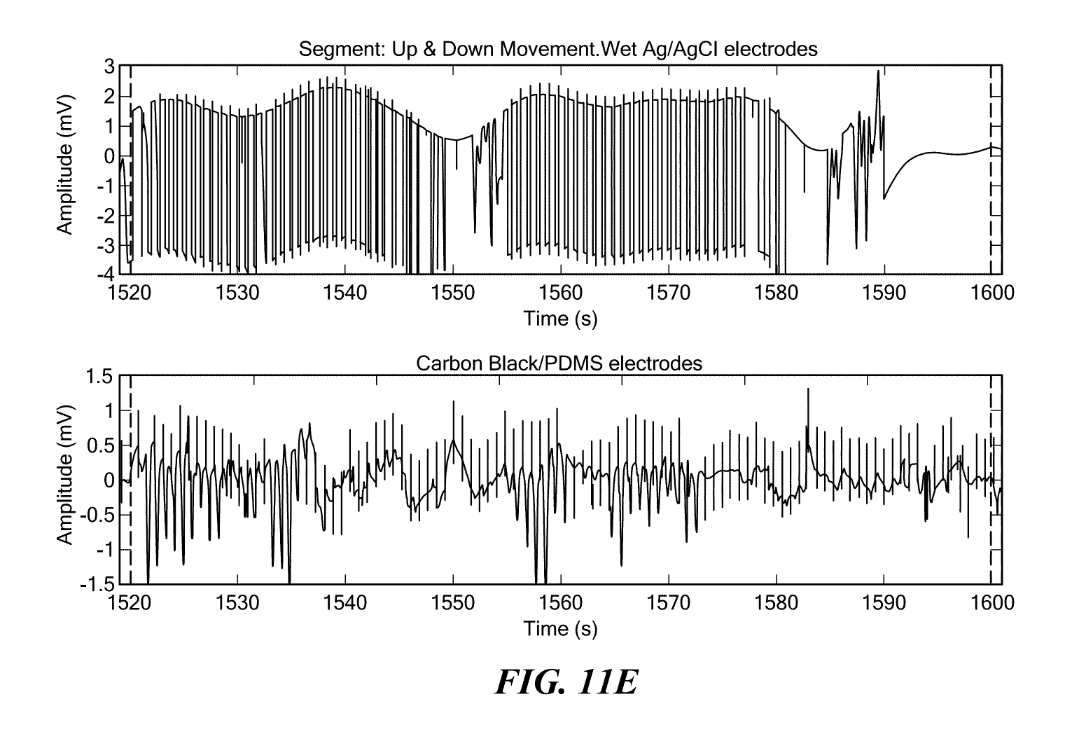

FIGS. 11A-11E depict ECG recording on surface and underwater with wet Ag/AgCl and Carbon Black/PDMS electrodes during different conditions, including with and without an elastic band applying pressure to the electrodes, in addition to adhesive tape. FIG. 11A is a full recording with aligned and filtered ECG signals. Dry condition are for a subject outside water, standing. Immersed condition apply to a subject in water, seated. Wet condition depict a subject outside water, standing after having been immersed. FIG. 11B shows an outside, band removed segment: subject outside water without elastic band, standing. FIG. 11C shows an inside, band removed segment: subject inside water without elastic band, seating. FIG. 11D shows a sequence depicting torso movement inside water without elastic band, seating. FIG. 11E shows measurement during up and down movement inside water without elastic band, seating.

To fully compare the hydrophobicity of both types of electrodes, the elastic band was removed so that both set of electrodes remained attached to the body only with their respective adhesive tapes (FIG. 11A, arrow). The subjects were then fully immersed in a sitting position and were instructed to sit quiet (FIG. 11C) for 1 minute followed by moving their body torso up-and-down (FIG. 11D) and side-to-side manner (FIG. 12E). As shown in FIG. 11C, the Ag/AgCl electrodes' ECG signals are immediately compromised even during a quite sitting position whereas for the CB/PDMS electrodes, high fidelity data can be seen. The Ag/AgCl electrodes' signal quality becomes saturated and consequently all morphological waveforms of the ECG are not discernible with both up-and-down and side-to-side movements as shown in FIG. 11D-E. Consequently, heart rate calculations cannot be performed. However, even with significant motion artifacts, the CB/PDMS electrodes are able to resolve QRS complexes, as known for ECG readings, throughout the data collection with body movements. There are visible low frequency oscillations which are due to cyclical body movements but they can be filtered to reveal all morphological waveforms of the ECG. The average heart rate computed via an automatic R-peak detection algorithm is presented in TABLE I for both types of electrodes for the ECG signal showed in FIGS. 11A-11E. For the side-to-side torso movements, the mean HR value from the Ag/AgCl are about the half the value of the CB/PDMS. For the up-and-down torso movements, heart rate calculations cannot be determined for the Ag/AgCl electrodes since we obtain saturated values throughout the recording. For CB/PDMS electrodes, we obtain similar HR values as those at other conditions including at rest period. Table I shows an average heart rate computed for the different conditions of the recording showed in FIGS. 11A-11E.

TABLE I

| | Wet Ag/AgCl | CB/PDMS |
|---|---|---|
| | electrodes | electrodes |
| | Average Heart | Average Heart |
| Condition | Rate (bpm) | Rate (bpm) |
| Dry | 76.6 | 77.2 |
| Immerse | 71.7 | 71.2 |
| Wet | 79.6 | 78.4 |
| Outside Water/Band Removed | 76.3 | 75.6 |
| Inside Water/Band Removed | 63.86 | 76.5 |
| Torso Movement | 37.03 | 75.8 |
| Up & Down Movement | — | 75.3 |

In a particular configuration, an environmentally independent electrode for an ECG (electrocardiogram) comprising a 20:1 carbon black to PDMS mass ratio may be formed by the following steps:

1. Conductive carbon black powder (commercially available as CD Carbon Black Super P Conductive, Alfa Aesar; Ward Hill Mass.) was dispersed into room temperature Polydimethylsiloxane, PDMS (commercially available as Sylgard® 184, Dow Corning Corporation; Auburn, Mich.), which was used as the insulating matrix.

2. C6H14 (hexane) was utilized as a solvent to mix the carbon black with the PDMS and optionally, with Trifluropropyl POSS (commercially marketed as FL0578, by Hybrid Plastics®), also known as FPOSS. The solution was mixed by hand for 60 seconds to distribute the particles.

3. The hexane/carbon black solution was then added to the PDMS and placed in an ultrasonic cleaner over a period of time and checked at 1-hour intervals. Depending on the volume of Hexane used times will vary—for 20 mL of Hexane 150 minutes may be effective.

4. The carbon black/PDMS mixture was then mixed with the PDMS curing agent (Included with Sylgard® 184) in a 10:1 mass ratio. In the example configuration, the ratio applies to the mass of PDMS only, not the carbon black.

5. The carbon black/PDMS/curing agent mixture was poured and leveled with a straight metal edge into wells forming disks within the electrode molds.

6. The mixture was applied to the reverse side of nickel-plated snap fasteners appropriately sized for terminals used as a monitor connection.

7. All components were degassed for 15 minutes in a vacuum chamber to remove air bubbles.

8. The fasteners/terminals are affixed/inserted to the molded carbon black/PDMS/curing agent mixture and placed on to the surface with gentle pressure without causing major rippling.

9. A final layer of PDMS/curing agent solution was mixed (following an accepted PDMS casting protocol) and no more than 0.5 g was poured into the top of the mold as a backing to the electrode. It should be noted that excessive PDMS might cover the fastener/terminal, thus preventing a connection to a monitor.

10. The filled mold assembly was then placed in a curing oven at 70° C. for 12 hours.

11. After the 12 hours the molds were disassembled and the electrodes removed.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A surface mountable electrode comprising:
a conductive substrate having a substantially planar sensing area adapted for communication with an electrically sensitive surface;
a terminal for connection to a monitor circuit, the terminal having electrical continuity with the planar sensing area;
the planar sensing area defining an impedance with a sensing surface conducive to electrical monitoring; and
the conductive substrate being flexible for electrical communication upon surface placement on the electrically sensitive surface.

2. The electrode of claim 1 wherein the defined impedance is independent of environmental conditions on the sensing surface.

3. The electrode of claim 2 wherein the defined impedance is substantially constant in wet or dry ambient conditions on the sensing surface.

4. The electrode of claim 2 wherein the conductive substrate includes a composition of a polymer and conductive particles.

5. The electrode of claim 4 wherein the conductive substrate includes a dispersion of carbon black such that the carbon black achieves a density based on a predetermined concentration defined by an ability to conduct an electrical signal through the substrate.

6. The electrode of claim 2 wherein the terminal is mounted to the substrate for connection to a control circuit, the control circuit responsive to the electrode, the electrode adapted to sense electrical signals unaffected by liquid presence on the substrate.

7. The electrode of claim 1 wherein the sensing surface is capable of carrying electrical signals sensed on the electrically sensitive surface from the sensing surface to the monitor circuit.

8. The electrode of claim 7 wherein the sensing surface is adapted to maintain a substantially constant impedance with the electrically sensitive surface independently of a mounting environment.

9. The electrode of claim 8 wherein the mounting environment is a human epidermis and the electrode is unaffected by sweat and water.

10. The electrode of claim 9 wherein the mounting environment is submerged underwater and the electrode is affixed to a cardiac region of a subject, the cardiac region for transmitting cardiac rhythms to the sensing circuit via the electrode.

11. The electrode of claim 2 wherein the conductive substrate is a homogeneous structure having a planar contact surface and an integrated electrode, the homogeneous structure formed around the terminal for passing electrical signals from the planar contact surface through the electrode to the control circuit.

12. The electrode of claim 2 wherein the defined impedance of the substrate is proportional to the area of the planar surface, such that the strength of a sensed electrical signals increases with the area.

13. A method for fabricating a surface mount electrode comprising the steps of:
combining a polymeric compound with a conductive medium;
adding a solvent to form a fluidic composition adapted to formation in a mold;
forming the composition into substantially planar shapes having a planar sensing area responsive to electrical signals on a sensing surface; and
inserting a terminal into the formed planar shape, the terminal configured for electrical connection to a monitor circuit.

14. The method of claim 13 wherein the composition is adapted to form conductive agglomerations based on a density of the dispersed conductive medium.

15. The method of claim 14 wherein the polymeric compound includes PDMS and the conductive medium is carbon black powder.

16. The method of claim 13 further comprising dissolving a predetermined quantity of Trifluropropyl POSS (FPOSS) into 50 ml of Asahiklin; and
adding the FPOSS solution to the composition.

17. The method of claim 16 wherein the predetermined quantity of FPOSS is in the range of 5-50 mg FPOSS per 50 ml of Asahiklin.

18. The method of claim 14 further comprising affixing the electrodes to underwater divers and receiving signals indicative of a respiration of the underwater diver.

19. The method of claim 18 further comprising monitoring the signals received from the underwater divers for detecting symptoms of decompression sickness (DCS)., i.e. bloodstream borne gas bubbles.

20. The method of claim 19 further comprising computing a heart rate variability (HRV) based on variances of distance between the peaks of the monitored signals, and identifying DCS based on the variances

21. The method of claim 14 further comprising
affixing an electrode to an underside of a wristwatch appliance in communication with the wrist epidermis for sensing cardiac signals;
affixing a complementary electrode on an epidermis of an opposed wrist for sensing a complementary signal; and
performing continuous monitoring of the cardiac signals obtained via the electrode and complementary electrode; and
storing the monitored signals in the wristwatch for subsequent analysis.

22. The method of claim 21 wherein the monitored signals detect and define continuous measurement of paroxysmal arterial fibrillation (abnormal heartbeat).

23. A method for fabricating a surface mount electrode comprising the steps of:
dispersing conductive carbon black powder into room temperature polydimethylsiloxane (PDMS), the PDMS providing an insulating matrix;
combining C6H14 (hexane) as a solvent to mix the carbon black with the PDMS to distribute particles of the resulting solution;
placing the hexane/carbon black solution and PDMS in an ultrasonic cleaner;
mixing a curing agent in a 10:1 mass ratio;
pouring and leveling the mixture with a straight metal edge into wells forming disks within the electrode molds;
applying the mixture to a reverse side of nickel plated snap fasteners appropriately sized for monitor connection;
degassing in a vacuum chamber to remove air bubbles;
affixing to the molded carbon black/PDMS/curing agent mixture by placing the electrodes on the surface with gentle pressure without causing major rippling;
mixing a final layer of PDMS/curing agent solution was mixed and pouring into the top of the mold as a backing to the electrode;
curing in an oven at 70° C. for 12 hours.
